(19) United States
(12) Patent Application Publication
(10) Pub. No.: US 2008/0146839 A1
(43) Pub. Date: Jun. 19, 2008

(54) Method for Producing 5-Halo-2,4,6-Trifluoroisophthalic Acid

(75) Inventors: Michael Rack, Eppelheim (DE); Sebastian Peer Smidt, Mannheim (DE); Manuel Budich, Bohl-Iggelheim (DE); Volker Maywald, Ludwigshafen (DE); Michael Keil, Freinsheim (DE); Bernd Wolf, Fussgonheim (DE)

Correspondence Address: HUTCHISON LAW GROUP PLLC, PO BOX 31686, RALEIGH, NC, 27612, US

(73) Assignee: BASF Aktiengesellschaft, Ludwigshafen (DE)

(21) Appl. No.: 11/908,870

(22) PCT Filed: Mar. 16, 2006
(86) PCT No.: PCT/EP2006/060793

§ 371 Date: Sep. 17, 2007

(30) Foreign Application Priority Data
Mar. 18, 2005 (DE) 10 2005 013 038.0

(51) Int. Cl. C07C 63/70; C07C 25/13

(52) U.S. Cl. 562/493; 570/142

(57) ABSTRACT

The invention relates to a method for producing 5-halo-2,4,6-tifluoroisophthalic acid of formula (I), wherein X represents F, Cl, Br, or I, by hydrolysis of 5-halo-2,4,6-trifluoroisophthalodinitrile of formula (II). Said invention is characterised in that in a first step, isophthalodinitrile (II) or a solution containing isophthalodinitrile (II) is reacted with a concentrated sulphuric acid at room temperature in order to form a 5-halo-2,4,6-trifluoroisophthalodiamide of general formula (III), and is, subsequently, heated and in a second step, isophthalic acid (I) is produced after additional heating and addition of water.

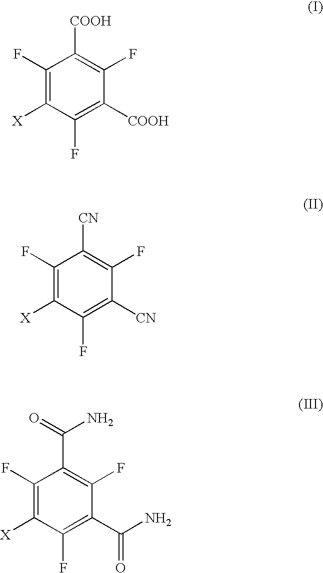

The present invention relates to a process for preparing 5-halo-2,4,6-trifluoroisophthalic acid of the formula I

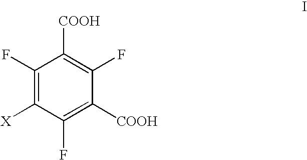

where X is F, Cl, Br, or I
by hydrolysis of 5-halo-2,4,6-trifluoroisophthalonitrile of the Formula II

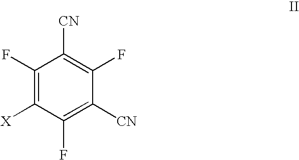

5-halo-2,4,6-trifluoroisophthalic acid (I) is an intermediate in the synthesis of trifluorobenzene, an important building block for the preparation of active ingredients in the medicaments and crop protection sector.

From the literature it was known (JP 62,111,942) that tetrafluorinated ortho-dicyanobenzenes can be converted by reaction in concentrated sulfuric acid and subsequent hydrolysis of the resulting intermediate in dilute sulfuric acid with high yield to the corresponding phthalic acid.

It was also known from the literature that halogenated meta-dicyanobenzenes can be hydrolyzed both in alkaline medium and with strong mineral acids to the corresponding isophthalic acids. At alkaline pH values, exchange reactions of halogen with hydroxide ions are observed. Owing to the particular substitution on the phenyl ring, the hydrolysis in an acidic reaction medium requires severe conditions.

Thus, U.S. Pat. No. 4,647,411 discloses a process in which tetrafluoroisophthalonitrile is hydrolyzed in 70% by weight sulfuric acid at from 157 to 162° C. in 15 h with a yield of 95% to tetrafluoroisophthalic acid. According to Kogyo Kagaku Zasshi (1979), 73(2), 447-8, 5-chloro-2,4,6-trifluoroisophthalonitrile is converted with 60% sulfuric acid under reflux within 5 hours to an extent of 78% to 5-chloro-2,4,6-trifluoroisophthalic acid. EP-A 1 256 564 teaches that 5-chloro-2,4,6-trifluoroisophthalic acid is obtained within 3 h by hydrolysis in 10 times the amount of 62% sulfuric acid heated to reflux from 5-chloro-2,4,6-trifluoroisophthalonitrile with 95.4% yield. The harsh reaction conditions of the disclosures U.S. Pat. No. 4,647,411, Kogyo Kagaku Zasshi (1979), 73(2), 447-8 and EP 1 256 564 are disadvantageous for industrial scale applications. At reaction temperatures of T>150° C., as in the presence of 62% by weight sulfuric acid heated to reflux, all common reactor materials are unstable.

It can be discerned directly from this that the prior art does not disclose any procedure employable on the industrial scale.

It was an object of the present invention to provide an economically viable process for preparing 5-halo-2,4,6-trifluoroisophthalic acid.

It was a particular object of the present invention to provide a process for preparing 5-halo-2,4,6-trifluoroisophthalic acid which features gentle reaction conditions and enables a good space-time yield.

Accordingly, a process has been found for preparing 5-halo-2,4,6-trifluoroisophthalic acid of the general formula I by hydrolysis of 5-halo-2,4,6-trifluoroisophthalonitrile of the general formula II, in which, in a first step to form 5-halo-2,4,6-trifluoroisophthalamide of the formula III,

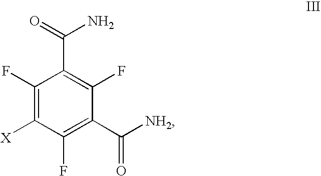

isophthalonitrile (II) or a solution comprising isophthalonitrile (II) is admixed with concentrated sulfuric acid at room temperature and subsequently heated, and, in a second step, isophthalic acid (I) is prepared with further heating and addition of water.

Definition of the variable:

X is halogen, i.e. fluorine, chlorine, bromine or iodine.

The process according to the invention is preferably employed to prepare compounds in which X is chlorine or bromine; X is more preferably chlorine.

According to the invention, isophthalonitrile (II) or a solution thereof is admixed in a first step with concentrated sulfuric acid. The isophthalonitrile (II) may be added in solid form to the concentrated sulfuric acid, for example in the form of powder or flakes. It is possible to introduce the isophthalonitrile (II) into the reaction in dissolved or water-moist form.

In one of the preferred embodiments, isophthalonitrile (II) is used in water-moist form. Water-moist is understood to mean residual water contents of preferably up to 40% by weight based on isophthalonitrile (II). Particular preference is given to introducing the compound of the formula II into the reaction with a water content of from 30 to 35% by weight.

When isophthalonitrile (II) is used in solid form, preference is given to preparing a suspension. Suspending means the very uniform distribution of the isophthalonitrile (II) solid in the concentrated sulfuric acid, for example by stirring. According to the invention, the isophthalonitrile (II) may also be introduced into the reaction dissolved in a solvent, for example as the product of value from a preceding process step.

The solvents used are, for example, aromatic solvents such as substituted or preferably unsubstituted alkylbenzenes such as methylbenzene, dimethylbenzenes or trimethylbenzenes, their isomer mixtures or chlorobenzenes. Particular preference is given to toluene.

According to the invention, concentrated sulfuric acid is used, generally in a concentration of at least 70% by weight. Preference is given to a sulfuric acid concentration of at least 80% by weight, particular preference to 90% by weight. When dissolved isophthalonitrile (II) is used, preference is given to using a sulfuric acid concentration of not more than 85% by weight. The amount of sulfuric acid in relation to the isophthalonitrile (II) is kept to a minimum and is generally less than 20 molar equivalents, for example from 3 to 20 molar equivalents, preferably from 4 to 10 molar equivalents, more preferably from 5 to 7 molar equivalents.

According to the invention, isophthalonitrile (II) or a solution thereof is admixed at room temperature with the concentrated sulfuric acid. In connection with the present invention, room temperature shall be understood to mean temperatures of below 50° C., in particular below 40° C. In general, the temperatures are above freezing point, preferably at 10° C. or higher. The temperature range is preferably from 20 to 30° C. Particular preference is given to a temperature range of from 25 to 30° C.

In the process according to the invention, both the isophthalonitrile (II) in solid (for example water-moist) or dissolved form and the sulfuric acid may be initially charged.

In one embodiment, the process according to the invention may be carried out under reduced pressure. In this case, the pressure is generally selected in such a way that the solvent used can be removed readily, for example by distillation.

The reaction is preferably conducted in such a way that the sublimation of isophthalonitrile (II) in the reaction system is substantially avoided, i.e. generally less than 0.5% by weight of the compound of the formula II sublimes.

In the process according to the invention, the sulfuric acid/isophthalonitrile (II) suspension or mixture is heated after full addition of the isophthalonitrile (II). The temperatures are preferably 110° C. or lower. A temperature range from 90 to 110° C. has generally been found to be advantageous. Particular preference is given to a temperature range of from 90 to 100° C., special preference to from 95 to 100° C. In this process step, isophthalamide (III) is formed as an intermediate. In one embodiment, the isophthalamide (III) is formed partially. Particular preference is given to reaction conditions under which the isophthalamide (III) is formed virtually quantitatively from the compound of the formula II.

In the process according to the invention, water is preferably added in a subsequent step at such a rate that, as a result of the exothermic reaction, the reaction mixture is not heated significantly above the temperatures specified below.

In general, the temperatures are in the range from 90 to 140° C. Preference is given to a temperature range of from 110 to 130° C. Particular preference is given to carrying out the reaction at temperatures of from 115 to 125° C.

Water may be added to the reaction, for example, by pouring, dripping or spraying. The temperature of the water added is not significant for the reaction; it is possible to add either cold or warm water to the reaction.

The hydrolysis to the isophthalic acid (I) is generally carried out with 3 or more molar equivalents. In general, 25 molar equivalents of water are sufficient. Preference is given to from 15 to 25 molar equivalents, particular preference to from 15 to 22 molar equivalents.

Usually, the reaction mixture is allowed to continue to react; for example, it is stirred at temperatures of from 90 to 140° C. for a further 2 to 12 h. In addition, preference is given to a reaction temperature of generally from 110 to 130° C. Particular preference is given to reaction temperatures of from 115 to 125° C. The reaction is continued until the reactants have reacted substantially, preferably fully, for example at least to an extent of 95% of theory. This may have been achieved, for example, as early as after 6 h.

At these relatively low temperatures, the reactor material is protected, i.e. reactors lined with fluoropolymer withstand these reaction conditions.

The reaction also proceeds at higher temperatures, but experience has shown that the wear of the reactor material increases without an improvement in the product quality being achieved.

The reaction steps of the process according to the invention may be carried out spatially separately or in one reactor. They are preferably carried out in one reactor in what is known as a one-pot process.

The process according to the invention can be carried out in such a way that isophthalamide (III) can be isolated. Methods for isolating isophthalamide (III) are known per se to those skilled in the art, or the isolation can also be undertaken by methods known to those skilled in the art.

However, preference is given to carrying out the process according to the invention up to the end product of the general formula I, 5-halo-2,4,6-trifluoroisophthalic acid, in a one-pot process without isolation of the compound of the formula III.

The isophthalamide (III) may also be used directly and hydrolyzed in dilute mineral acid to the 5-halo-2,4,6-trifluoroisophthalic acid. To this end, preference is given to using sulfuric acid in a concentration range from 30 to 80% by weight, more preferably from 40 to 70% by weight sulfuric acid.

Isophthalamide (III) is hydrolyzed generally with from 3 to 18 molar equivalents of sulfuric acid. Preference is given to using from 4 to 8 molar equivalents, particular preference to using from 4 to 5 molar equivalents. Preference is further given to a reaction temperature in the range from 110 to 130° C. Particular preference is given to carrying out the reaction at temperatures of from 115 to 125° C.

In general, the reaction mixture is stirred at temperatures of from 115 to 125° C. for from 2 to 8 h. The reaction is continued until the reactants have reacted substantially, preferably fully, for example at least to an extent of 95%. This may be achieved, for example, as early as after 6 h.

It is possible to work up the reaction product by extracting it from the reaction solution with organic solvents; examples of advantageously suitable solvents are methyl tert-butyl ether, ethyl tert-butyl ether and ethyl or propyl acetate.

The compound of the formula I can be used as an intermediate starting from the compound of the formula II in a decarboxylation reaction to prepare 2-halo-1,3,5-trifluorobenzene of the formula IV, as described, for example, in EP-B1 460 639 (example 1, page 5, lines 32-47). Preference is given to carrying out the decarboxylation by heating the compound of the general formula I in a polar solvent with or without addition of catalysts at temperatures between 110 to 250° C.

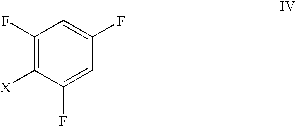

The compound of the general formula IV can be used as an intermediate starting from the compound of the formula II in a dehalogenation reaction to prepare 1,3,5-trifluorobenzene of the formula V, as described, for example, in EP-B1 460 639 (example 2, page 5 and example 3, page 6). Preference is given to carrying out the dehalogenation by heating the compound of the general formula IV in the presence of a metal and water under pressure at temperatures between 100 to 200° C.

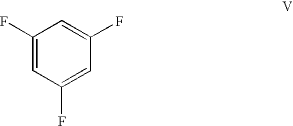

The present process is notable not only in that it enables the gentle industrial scale preparation of isophthalic acid (I), but also in that the volume of sulfuric acid required is low. This is particularly advantageous with regard to the isolation of the product of value from the reaction mixture and the disposal of the residues.

The process according to the invention proves to be advantageous from a process technology point of view by the possibility of being able to introduce the compound of the formula II into the reaction dissolved in a solvent.

Further advantages of the inventive reaction are the high space-time yield and a small by-product spectrum.

### COMPARATIVE EXAMPLES

### Comparative Example 1

According to Kogyo Kagaku Zasshi (1979), 73(2), 447-8:

2.0 g of 5-chloro-2,4,6-trifluoroisophthalonitrile and 10 ml of 60% sulfuric acid are heated under reflux (approx. 170° C.) over a period of 5 hours. After cooling, the precipitated crystals are filtered off, washed with 18% hydrochloric acid and dried; this affords 1.82 g of carboxylic acid (yield=78% of theory, mp=202 to 203° C.).

### Example 2

According to EP 1256 564:

5-chloro-2,4,6-trifluoroisophthalonitrile was hydrolyzed in 10 times the amount of 62% H₂SO₄ to 5-chloro-2,4,6-trifluoroisophthalic acid at 170° C. (reflux) over 3 h. Purity according to ¹⁹F NMR analysis: 90%, yield 86%. The reaction was also carried out at 150° C. (72% of theory) and at 130° C. (83% of theory). The by-product spectrum was larger at low temperatures than at higher temperatures.

### INVENTIVE PROCESS EXAMPLES

### Example 1

197 g (0.88 mol) of 5-chloro-2,4,6-trifluoroisophthalonitrile was suspended at room temperature in 624.5 g (6.37 mol) of 96% by weight H₂SO₄ in a glass round-bottom flask and subsequently heated to 100° C. 327.6 g (18.18 mol) of water were added dropwise at such a rate that the reaction mixture was heated to 120° C. and stirs at 120° C. for a further 8 h. After cooling, the reaction mixture was stirred into 2000 ml of cold water and extracted twice with 500 ml of methyl tert-butyl ether (MTBE), and the combined organic phases were dried and concentrated under reduced pressure. 228.1 g of 5-chloro-2,4,6-trifluoroisophthalic acid were obtained as a beige solid. Purity according to ¹⁹F NMR: 94% (96.2% of theory).

### Example 2

A suspension was prepared at room temperature from 72.3 g of water-moist 5-chloro-2,4,6-trifluoroisophthalonitrile (approx. 0.23 mol; 30% by weight of water) and 164 g (1.62 mol, 7 equivalents) of concentrated sulfuric acid (95-97% by weight). The resulting suspension was heated to 100° C. 61 ml (3.39 mol, 15 equivalents) of water were added at such a rate that a temperature of 122° C. was attained. Subsequently, the mixture was stirred at 120° C. for 8 hours. After cooling, 300 g of water were added and the internal temperature was kept below 45° C. The mixture was extracted twice with 85 g each time of MTBE. The two organic phases were combined and washed once with 50 ml of water, stirred with activated carbon and sodium sulfate, and filtered. The slightly yellowish organic phases were concentrated. 62.5 g of beige diacid (I) were obtained.

### Example 3

185.7 g (1.62 mol) of sulfuric acid (85% by weight) were initially charged and heated to 30° C. Subsequently, a solution of 50 g (0.46 mol) of 1-chloro-2,4,6-trifluoroisophthalonitrile in 100 ml of toluene was added dropwise at 30-35° C. under reduced pressure. Toluene was distilled off continuously. Subsequently, the reaction mixture was heated to 100° C. 61.1 g (3.39 mol; 7.4 equivalents) of water were added. Subsequently, the mixture was heated to 130° C. and stirred for 2 h. After cooling to 60° C., first 150 ml of water were added. The fine suspension was extracted twice with 100 ml each time of MTBE. The organic phases were combined, stirred with activated carbon and sodium sulfate, and filtered. The slightly yellowish organic phases were concentrated. 59 g of beige diacid (I) were obtained.

1-13. (canceled)

14. A process for preparing a 5-halo-2,4,6-trifluoroisophthalic acid of formula I: 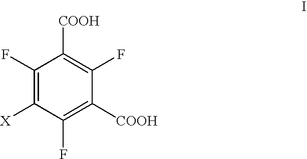
wherein X is F, Cl, Br, or I,
the process comprising the steps of:
a) hydrolyzing a 5-halo-2,4,6-trifluoroisophthalonitrile of formula II: 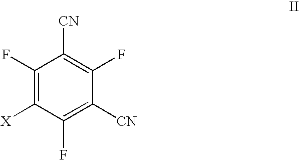
by admixing the 5-halo-2,4,6-trifluoroisophthalonitrile of formula II, or a solution comprising the 5-halo-2,4,6-trifluoroisophthalonitrile of formula II, with concentrated sulfuric acid at room temperature, and subsequently heating the 5-halo-2,4,6-trifluoroisophthalonitrile of formula II and the concentrated sulfuric acid up to 110° C. to yield a 5-halo-2,4,6-trifluoroisophthalamide of formula III: 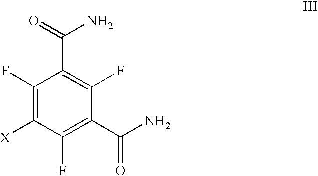
b) heating the 5-halo-2,4,6-trifluoroisophthalamide of formula III to 110 to 130° C. and adding water to yield the 5-halo-2,4,6-trifluoroisophthalic acid of formula I.

15. The process according to claim 14, wherein heating is effected in step a) to 90to 110° C.

16. The process according to claim 14, wherein the 5-halo-2,4,6-trifluoroisophthalonitrile of formula II is suspended in the concentrated sulfuric acid.

17. The process according to claim 14, wherein the 5-halo-2,4,6-trifluoroisophthalonitrile of formula II is used in water-moist form.

18. The process according to claim 14, wherein the 5-halo-2,4,6-trifluoroisophthalonitrile of formula II is introduced into the reaction dissolved in a solvent.

19. The process according to claim 14, wherein step a) is carried out with an amount of at least 3 equivalents of water, based on the 5-halo-2,4,6-trifluoroisophthalonitrile of formula II.

20. The process according to claim 14, wherein steps a) and b) are carried out in a one-pot process.

21. The process according to claim 14, further comprising the step of isolating the 5-halo-2,4,6-trifluoroisophthalamide of formula III.

22. The process according to claim 21, wherein the isolated 5-halo-2,4,6-trifluoroisophthalamide of formula III is heated to 110 to 130° C. and water is added to yield the 5-halo-2,4,6-trifluoroisophthalic acid of formula I.

23. A process of preparing a 2-halo-1,3,5-trifluorobenzene of formula IV, 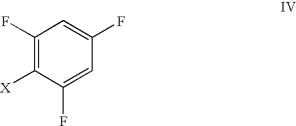
the process comprising:
decarboxylating a 5-halo-2,4,6-trifluoroisophthalic acid of formula I: 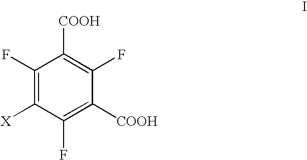
wherein X is F, Cl, Br, or I.

24. The process according to claim 23, wherein the 5-halo-2,4,6-trifluoroisophthalic acid of formula I is prepared by the steps of:
a) hydrolyzing a 5-halo-2,4,6-trifluoroisophthalonitrile of formula II: 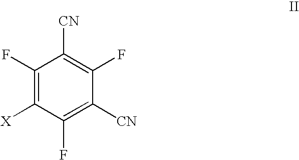
by admixing the 5-halo-2,4,6-trifluoroisophthalonitrile of formula II, or a solution comprising the 5-halo-2,4,6-trifluoroisophthalonitrile of formula II, with concentrated sulfuric acid at room temperature, and subsequently heating the 5-halo-2,4,6-trifluoroisophthalonitrile of formula II and the concentrated sulfuric acid up to 110° C. to yield a 5-halo-2,4,6-trifluoroisophthalamide of formula III: 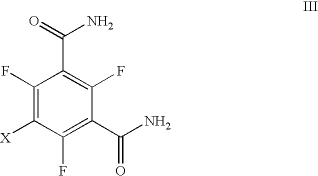
b) heating the 5-halo-2,4,6-trifluoroisophthalamide of formula III to 110 to 130° C. and adding water to yield the 5-halo-2,4,6-trifluoroisophthalic acid of formula I.

25. A process for preparing a 1,3,5-trifluorobenzene of formula V, 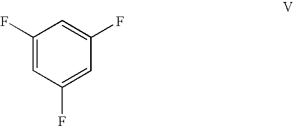
the process comprising:
a) decarboxylating a 5-halo-2,4,6-trifluoroisophthalic acid of formula I 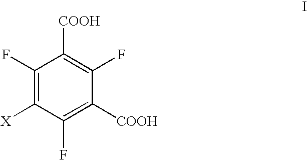
wherein X is F, Cl, Br, or I, to yield a 2-halo-1,3,5-trifluorobenzene of formula IV: 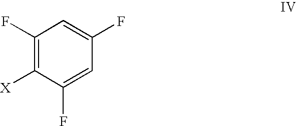
and subsequently
b) dehalogenating the 2-halo-1,3,5-trifluorobenzene of formula IV to yield the 1,3,5-trifluorobenzene of formula V.

26. The process according to claim 25, wherein the 5-halo-2,4,6-trifluoroisophthalic acid of formula I is prepared by the steps of:
a) hydrolyzing a 5-halo-2,4,6-trifluoroisophthalonitrile of formula II: 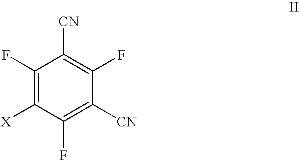
by admixing the 5-halo-2,4,6-trifluoroisophthalonitrile of formula II, or a solution comprising the 5-halo-2,4,6-trifluoroisophthalonitrile of formula II, with concentrated sulfuric acid at room temperature, and subsequently heating the 5-halo-2,4,6-trifluoroisophthalonitrile of formula II and the concentrated sulfuric acid up to 110° C. to yield a 5-halo-2,4,6-trifluoroisophthalamide of formula III: 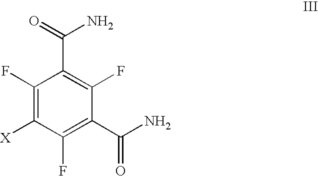
b) heating the 5-halo-2,4,6-trifluoroisophthalamide of formula III to 110 to 130° C. and adding water to yield the 5-halo-2,4,6-trifluoroisophthalic acid of formula I.
